(19) United States
(12) Patent Application Publication
(10) Pub. No.: US 2024/0350806 A1
(43) Pub. Date: Oct. 24, 2024

(54) OPTIMAL TARGET SELECTION FOR NON-INVASIVE NEUROMODULATION

(71) Applicant: Regents of the University of Minnesota, Minneapolis, MN (US)

(72) Inventors: Oscar Miranda Dominguez, Minneapolis, MN (US); Cristian Andres Morales Carrasco, III, Minneapolis, MN (US); Damien Fair, Minneapolis, MN (US)

(21) Appl. No.: 18/642,248

(22) Filed: Apr. 22, 2024

Related U.S. Application Data
(60) Provisional application No. 63/497,436, filed on Apr. 20, 2023.

(51) Int. Cl. A61N 1/36; A61N 2/00; G16H 15/00; G16H 40/63

(52) U.S. Cl. CPC A61N 1/36128; G16H 15/00; G16H 40/63; A61N 2/006

(57) ABSTRACT

Subject-specific energy distribution mapping of functional networks is used to inform the planning, guidance, and/or monitoring of neuromodulation, including non-invasive brain stimulation, deep brain stimulation, prefrontal cortical stimulation, intracranial electrical stimulation, focused ultrasound-based neuromodulation, pharmacological-based neuromodulation, or the like.

### BACKGROUND

Transcranial Magnetic Stimulation (TMS) is effective in treating disorders such as depression and obsessive compulsive disorders. TMS induces an electric field in a target region related to a behavior of interest to be modulated. Its effectiveness, however, varies across individuals, in part due to differences in functional neuroanatomy. Recent clinical trials have shown increased efficacy when using individualized anatomical and functional information about brain organization. Even when targets are precisely located, little is known about the spatial distribution of the electric field across the brain networks, information that is needed to understand the extent to which stimulation spreads to non-targeted areas.

### SUMMARY OF THE DISCLOSURE

It is an aspect of the present disclosure to provide a method for planning, guiding, or monitoring a delivery of neuromodulation to a subject's brain. The method includes accessing subject-specific energy distribution mapping data with a computer system. One or more target locations are determined in the subject-specific energy distribution mapping data using the computer system. The one or more target locations indicate locations to which neuromodulation should be delivered. The one or more target locations are localized relative to the brain of the subject, thereby planning, guiding, or monitoring delivery of the neuromodulation to the one or more target locations.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows an example workflow for end-to-end processing of image data to generate subject-specific energy distribution mapping data.

FIG. 2 is a flowchart of an example method for guiding or otherwise monitoring the delivery or administration of a neuromodulation therapy to a subject based on a subject-specific energy distribution maps.

FIG. 3 shows an example workflow for generating subject-specific energy distribution mapping data.

FIGS. 4A and 4B show examples of transcranial magnetic stimulation (TMS) informed by neuronavigation with individualized anatomy and brain connectivity using the methods described in the present disclosure.

FIG. 5 shows a detailed view of example subject-specific energy distribution maps generated using the methods described in the present disclosure.

FIG. 6 shows example whole-brain subject-specific energy distribution maps generated using the methods described in the present disclosure.

FIG. 7 shows example precision functional mapping data for four individuals.

FIGS. 8A-8C show example subject-specific energy distribution maps generated using the methods described in the present disclosure.

FIG. 9 is a block diagram of an example system for generating subject-specific energy distribution mapping data and performing neuromodulation planning and/or guidance using the methods described in the present disclosure.

FIG. 10 is a block diagram of example components that can implement the system of FIG. 9.

### DETAILED DESCRIPTION

Described here are systems and methods for maximizing or otherwise optimizing the amount of delivered energy in a target brain area after the application of different forms of energy intended to provide non-invasive neuromodulation in a way that minimizes the delivered energy on undesired brain functional networks. Examples of neuromodulation therapies include non-invasive brain stimulation such as transcranial magnetic stimulation (TMS), transcranial direct current stimulation (TCDS), transcranial alternating current stimulation (TCAS), or the like. Additionally or alternatively, neuromodulation therapy can include other neurostimulations (e.g., deep brain stimulation, prefrontal cortical stimulation, intracranial electrical stimulation), focused ultrasound-based neuromodulation, pharmacological-based neuromodulation, or the like.

In general, the disclosed methods may include the following steps. A brain area (e.g., cortical or subcortical area), is selected based on its potential therapeutic effect (e.g., the clinical team decides to stimulate the prefrontal cortex of the subject). A brain functional map that identifies the location of brain functional networks is calculated for the subject. This assignment may be based on: data from the same subject and derived using template matching or another clustering algorithm such as community detection, or publicly available age-specific atlases. As a non-limiting example, the methods described in co-pending U.S. Patent Application Publication No. US 2023/0115330, which is herein incorporated by reference in its entirety, may be used.

A personalized finite element model (FEM) model may be calculated for the subject based on data from the same participant or using publicly available age-specific atlases. This model simulates the way the energy delivered by the energy-delivering device (e.g., coil for TMS) travels from its location to the target brain area and is transduced into a type of energy with a clinical effect.

A series of computational simulations explore all the accessible brain areas for the subject for a given energy-delivering device to identify the optimal location and orientation of such device. Optimal energy may include the largest amount of energy being delivered to the brain area with potential therapeutic effect. Additionally or alternatively, optimal energy may include the lowest amount of energy dispersed on networks not belonging to the network the target brain area belongs to.

In a non-limiting example, the computational simulations may be implemented using simnimbs-cifti-tools (SCT), a software developed that builds on the existing SimNIBS (simulation of non-invasive brain stimulation) software tool described by A. Thielscher, et al., in “Field modeling for transcranial magnetic stimulation: a useful tool to understand the physiological effects of TMS?” IEEE EMBS 2015, explores all the accessible brain areas, projects the calculated transduced energy to subject-specific cortical surfaces, quantifies the amount of energy delivered to each brain area and network, and selects the optimal target brain area according to the defined or otherwise selected optimization criteria.

SCT is a modeling framework designed to provide users with optimal brain targets for TMS, or other neuromodulation, interventions. It integrates SimNIBS with modern MRI data collection protocols and processing streams and performs exhaustive data-mining to identify optimal targets and their corresponding coil position and orientation. The outputs can include summary tables, an executive summary, and auxiliary figures. The SCT framework utilizes anatomical and functional MRI data per participant. Those images can be collected in advance to any planned clinical intervention. Examples of data collection and processing workflows are described below.

The SCT framework described in the present disclosure uses functional and structural (i.e., anatomical) magnetic resonance image data to model how the electromagnetic energy delivered by neuromodulation is induced in electric fields across the brain and other tissue and media (e.g., air and cerebral spinal fluid (CSF)).

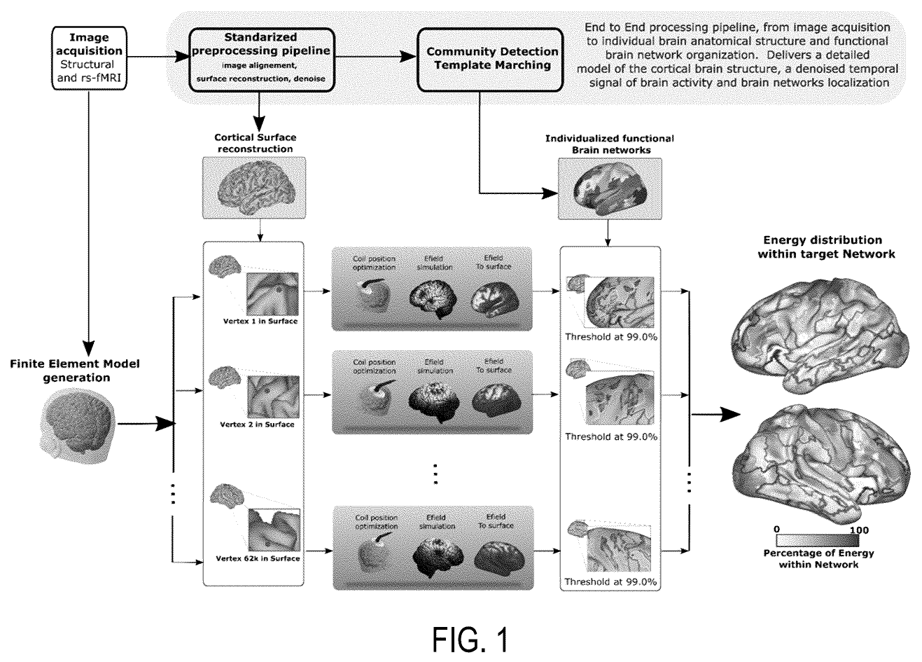

FIG. 1 shows an example workflow for an end-to-end processing pipeline for optimal target selection for non-invasive neuromodulation using the disclosed SCT framework. Image data are acquired, which may include structural image data (e.g., MPRAGE images, T1-weighted images, T2-weighted images, etc.) and functional image data (e.g., resting state fMRI (rs-fMRI) data, or task-based fMRI data). As will be described, the image data may be used as an input to a finite element model (FEM) for generating patient-specific energy distribution maps for within target networks. The image data may also be used to generate cortical surface reconstructions, and to generate individualized functional brain networks, as will also be described.

The structural and/or functional image data may be preprocessed to obtain coordinates and/or grayordinates. A grayordinate is a brainordinate within the gray matter of a subject's brain, and a brainordinate is a coordinate (e.g., a particular location) within a subject's brain. As one example, a brainordinate can be specified by a surface vertex, or node. As another example, a brainordinate can be specified by a volume voxel. Thus, a grayordinate corresponds to a particular location in the gray matter that can be specified as gray-matter surface vertices (e.g., cortical gray matter), gray-matter volume voxels (e.g., subcortical gray matter), or both. From the functional image data, timeseries data can be created for voxels that depict blood-oxygen-level dependent (BOLD) signals. The BOLD data can be projected to a surface reconstruction (mesh) of the cortex. Time course signals are created for each grayordinate.

As mentioned above, SCT framework may implement finite element modeling (e.g., using SimNIBS or other techniques), coil optimization for each coordinate and/or grayordinate (e.g., using ADM in SimNIBS, or other techniques), electrical field modeling for each target using the optimal coil position (e.g., using SimNIBS or other techniques), and converting the results into surface space (e.g., using SimNIBS or other techniques). Areas where the incident electrical field reaches a threshold level of peak applied energy are thresholded and measured. For example, the threshold level may be 99.0% of the peak applied energy. The networks where the surviving area lies (e.g., networks from template matching) are identified. The number of coordinates per network with grayordinates above the threshold incident electrical field are counted. The percent of incident energy within the network is then assigned to the target grayordinate. The workflow may also include an atlas generation step (e.g., by generating a probabilistic map of energy distribution within networks).

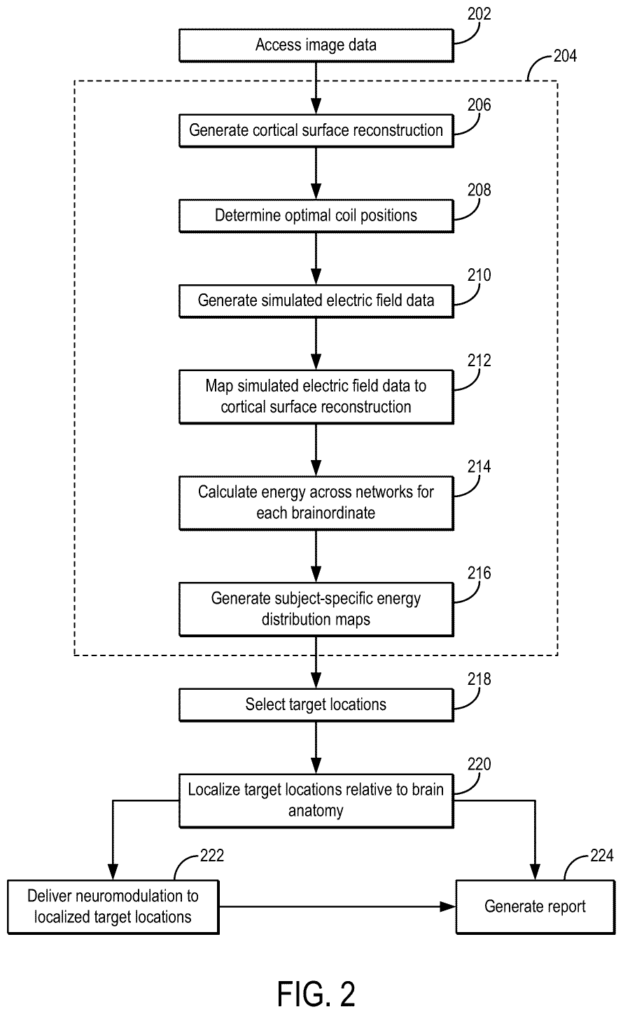

Referring now to FIG. 2, a flowchart is illustrated as setting forth the steps of an example method for guiding or otherwise monitoring the delivery or administration of a neuromodulation therapy to a subject based on a subject-specific energy distribution maps.

In the proposed processing pipeline, the optimal coil position to maximize the energy delivered in a given functional network is identified, where the networks are defined per subject and further parcellated anatomically by brain lobe and hemisphere. The target is a functional network instead of a particular brain region. A full exploration of cortical areas within the target network is performed. In alternative implementations, the search may be constrained to cortical areas on the network's gyral crown. By doing a full exploration, more coil positions and orientations that could target cortical areas whose stimulation maximizes the energy delivered within the network can be identified. Network delineation and their corresponding energy distribution are estimated using personalized models informed by functional and structural MRI, respectively. Hence, the pipeline requires anatomical and functional resting-state brain images, and can generate output as coil position data ready to be used by a neuronavigation system in the clinic setup.

As will be described, the pipeline makes use of precision functional mapping (PFM) acquisition and preprocessing pipelines, as well as community detection algorithms to identify functional networks. As a non-limiting example, the pipeline can be built on top of electric field simulation software (e.g., SimNIBS), which builds an individualized finite element model of the participant head based on their T1 and T2 anatomical images and calculates the electric field propagation on the tissue for any coil position the user provides.

The pipeline presented here can be used to optimize the coil position for a given intervention or planning to take into account the individualized functional brain organization.

The method includes accessing image data with a computer system, as indicated at step 202. Accessing the image data can include retrieving previously generated data from a memory or other data storage device or medium. Additionally or alternatively, accessing the image data can include acquiring the data with an MRI system. As described above, the image data may include structural image data (e.g., anatomical images acquired using MRI) and functional image data. The functional image data may include rs-fMRI image data, or maps generated therefrom. Maps generated from functional image data may include individual-specific functional network maps and/or individual-specific integration zone maps.

Structural image data are used to identify tissues and the functional data to delineate brain areas and networks. Then, several subsets of neighboring brain areas are selected as potential targets for neuromodulation intervention. The analytic pipeline compares the performance of all the candidates and picks the best based on a predefined optimization criterion, as described below.

The functional image data (e.g., rs-fMRI data) can be processed to generate precision functional mapping (PFM) data. As a non-limiting example, precision brain mapping of functional neural networks is a technique for examining individual network topography. Network topography across individuals appears to have some shared features, but the general shape and strength of connections remains highly individual-specific. PFM data may include individual-specific network maps. As one example, the PFM data may include an individual-specific functional network map and/or an individual-specific integration zone map. There are a variety of techniques for generating PFM data, such as template matching and InfoMap. For example, the methods described in co-pending U.S. Patent Application Publication No. US 2023/0115330, which is herein incorporated by reference in its entirety, may be used to create PFM data in some examples.

A functional network map can be generated using an overlapping template matching technique, or another suitable technique. In an overlapping template matching technique, a functional network map can be generated from functional network template data corresponding to a plurality of different functional networks. The functional network template data can include templates for the following functional networks: the default mode network (DMN), the visual network (VIS), the frontal parietal network (FPN), the dorsal attention network (DAN), the ventral attention network (VAN), the salience network (Sal), the cingulo-opercular network (CO), the sensorimotor dorsal network (SMD), the sensorimotor lateral network (SML), the auditory network (AUD), the temporal pole network (Tpole), the medial temporal network (MTL), the parietal occipital network (PON), and the parietal medial network (PMN). In other implementations, the templates can include fewer of these functional networks and/or can include additional functional networks.

As one example, a community detection method using template matching can be used to generate individual-specific functional network maps. In general, a template matching algorithm identifies, at the individual level, the functional brain networks based on highly sampled fMRI data. The outcome is the assignment of every cortical and subcortical grayordinates to a given functional network. For example, from the functional network template data, individual-specific network assignments can be determined. A voxelwise correlation matrix can be generated by correlating the BOLD signals for each grayordinate with the BOLD signals every other grayordinate represented in time course signal data acquired from the subject. The similarity between the correlation matrix and one or more of the templates in the functional network template data is then computed. Based on the similarity values, each grayordinate is assigned to one or more functional networks, generating output as one or more individual-specific functional network maps.

An integration zone map can be generated by determining the functional networks associated with each grayordinate in an individual-specific functional network map and assigning grayordinates in the integration zone map to one or more integration zones based on the functional networks associated with each grayordinate. As one example, the number of functional network assignments at each grayordinate can be counted, and the counts of overlapping functional networks at each grayordinate can be stored as the integration zone map. The count of networks at each grayordinate can define an integration zone. Additionally or alternatively, the specific functional networks assigned to each grayordinate can also be stored for each grayordinate in the integration zone map. In some embodiments, the number of networks and specific networks at each grayordinate can collectively define different integration zones. For example, clusters of grayordinates associated with overlapping functional networks can be identified and integration zones can be defined based on those identified clusters.

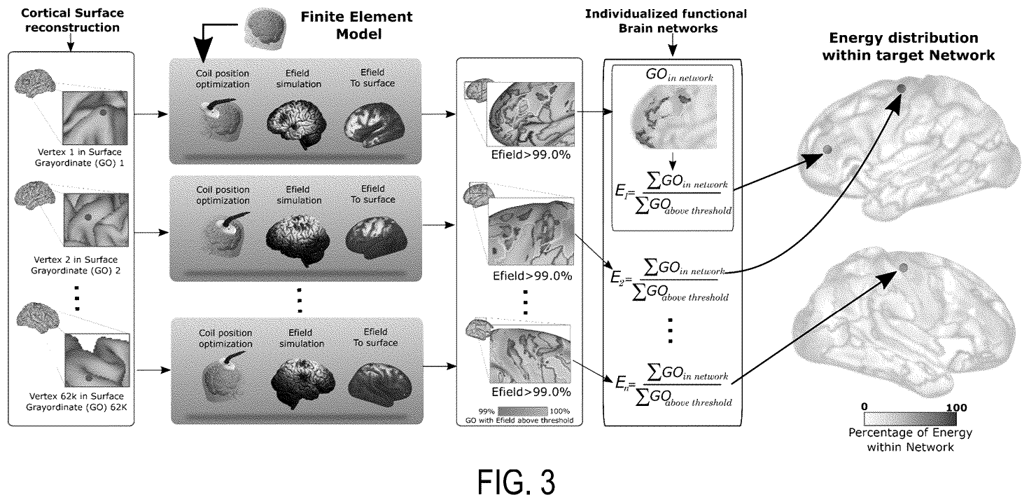

From the image data, subject-specific energy distribution maps may be generated with the computer system, as indicated at process block 204. For instance, the subject-specific energy distribution maps within one or more target networks may be generated using the framework outlined in FIG. 3, as described below. Using this patient-specific energy distribution, the optimal location and orientation of an energy-delivering device for neuromodulation can be identified.

In general, a cortical surface reconstruction is generated from the image data, as indicated at step 206. As described above, the cortical surface reconstruction may be generated from the structural image data. In some instances, the cortical surface reconstruction may include a mesh surface, a 3D volume, or the like. As a non-limiting example, an FEM can be generated from the structural image data. As a non-limiting example, T1-weighted and T2-weighted images can be coregistered. Then, the images can be segmented into various segments, such as scalp, skull, CSF, gray matter (GM), white matter (WM) and eyes can be segmented. The segmented regions can be identified and a corresponding segmentation mask is generated for each one of these tissues. A mesh can then be generated using the segmented regions, resulting in a surface and volumetric grid using elements (e.g., tetrahedral elements) to build the surface and volume of the subject's brain.

Optimal coil locations are then determined based on the cortical surface reconstruction, as indicated at step 208. For instance, an auxiliary dipole method (ADM) can be used to identify optimal coil locations for each grayordinate and/or brainordinate in the cortical surface reconstruction. In general, the coil locations can be determined by searching coil positions in a grid around a candidate location (e.g., candidate grayordinate or brainordinate) and changing the orientation of the coil until an optimal coil orientation is identified for the candidate location. Candidate locations can be selected based on grayordinates or other brainordinates identified from the functional image data (e.g., from individual-specific functional network maps, integration zone maps, or both).

As an example, a search for optimal coil locations is iterated across the whole brain grayordinates, or a subset of them selected for a specific functional brain network of interest. Grayordinate selection is carried on the cortical surface reconstruction. For each grayordinate on the brain or the network of interest, the optimal coil position and orientation that maximize the electric field on the grayordinate is obtained using an ADM, or the like. The coordinates in Euclidean space (e.g., x,y,z coordinates) can be provided for any given grayordinate and the method calculates the optimal coil positioning and orientation that reaches the maximum intensity in the target.

Simulated electric field data are then generated based on the determined target locations (e.g., grayordinates or other brainordinates), as indicated at step 210. For instance, the electric field distribution for the target location and associated coil position (e.g., coil position and orientation at the grayordinate) can be calculated. The simulated electric field data are then mapped to the cortical surface based on the cortical surface reconstruction, as indicated at step 212. As an example, with the optimal coil position and orientation, electric field simulation is performed to generate a brain-wide electric field distribution on volume and projected to the cortical surface. The volumetric distribution can be converted into a surface distribution and then into a native space (e.g., a CIFTI space, which is the data format leveraged by the Human Connectome Project to organize brain functional data in a structured manner). This process can be iterated to obtain the electric field distribution when targeting each grayordinate, resulting in a coil position and electric field distribution dataset for the individual.

With the individualized brain networks and the electric field distribution (i.e., the simulated electric field data) calculated for all of the grayordinates in the cortical surface reconstruction, the energy is calculated across all the networks for each grayordinate, as indicated at step 214. The electric field is thresholded at a high value (99.9%) of the peak value and the number of grayordinates within each network are counted. In other implementations, a threshold less than 99.9% of the peak value can be used. In general, the relative electric field energy distribution map per network is dependent of the threshold used. In example studies, a high threshold value of 99.9% of the peak was chosen as a conservative value assuming only high values would induce an activation or inhibition in the cortical structures. In some implementations, the threshold may be selected using an electric field distribution probabilistic template, which indicates the probable expected relative electric field at each grayordinate.

The relative energy distribution can be calculated at each grayordinate (GO) as the ratio of the number of grayordinates surviving the threshold within the network (GO in network) and the total number of grayordinates surviving the thresholding (GO above threshold). The rationale for this index is to measure the amount of electric energy delivered to the target network is actually being delivered to the network and how much is being delivered to the entire brain (or surrounding networks). Thus, the relative electric field energy can then be calculated as the number of grayordinates within a network divided by the total surviving grayordinates above the threshold:

$${E}_{i} = \frac{\sum G {O}_{in \_ network}}{\sum G {O}_{above \_ threshold}} . \qquad ( 1 )$$

This calculation for each grayordinate results in an individualized map of relative energy distribution for all the participants, as indicated at step 216. The relative energy distribution can be calculated for each grayordinate in the cortex.

Based on the subject-specific energy distribution maps, one or more target locations to which neuromodulation therapy will be delivered are selected using the computer system, as indicated at step 218. The one or more target locations can be grayordinates or other brainordinates that are selected based on the condition for which neuromodulation therapy is being provided. Additionally or alternatively, a target location can include a group or cluster of grayordinates, such as a group or cluster of grayordinates associated with a common functional network, two or more different functional networks, or the like.

Thus, in general, selecting a target location for neuromodulation therapy can include determining a functional network, or networks, that when modulated by a neuromodulation therapy would provide a therapeutic effect to the subject (e.g., by controlling and/or improving the condition of the subject, or the like). As one example, target locations can be selected based on the condition to be treated. In some instances, selecting target locations can include selecting locations in the subject-specific energy distribution maps that have a probability of being assigned to a particular functional network that is above a certain threshold (e.g., 50%, 60%, 70%, 80%, 90%, etc.).

After the one or more target locations (e.g., grayordinates) to receive neuromodulation therapy are selected based on the subject-specific energy distribution maps, the target locations are localized within the subject, as indicated at step 220. For example, localizing the target locations can include identifying the target locations relative to the subject's anatomy, such that neuromodulation therapy can be delivered to the anatomical locations within the subject that correspond to the target locations selected relative to the subject-specific energy distribution maps.

In a non-limiting example, the target locations can be localized within the subject by accessing medical image data of the subject using the computer system, where the medical image data include, for example, at least one anatomical image depicting the brain of the subject to whom neuromodulation treatment will be delivered. As an example, the medical image data may be anatomical magnetic resonance images of the subject. Accessing such magnetic resonance images can include retrieving previously acquired images from a memory or other data storage device or medium. Additionally or alternatively, accessing the magnetic resonance images can include acquiring the images with an MRI system and transferring or otherwise communicating the data to the computer system, which in some embodiments may be a part of the MRI system.

The medical image data and the subject-specific energy distribution maps can then be coregistered, such that locations (e.g., grayordinates) in the subject-specific energy distribution maps can be associated with anatomical locations within the subject. In this way, the target locations selected in the subject-specific energy distribution maps can be localized relative to the subject's own anatomy.

Neuromodulation therapy is then delivered to the localized target location(s), as indicated at step 222. For example, neuromodulation therapies such as TMS, TDCS, and/or TACS can be delivered transcranially to the localized target location(s) by positioning the neurostimulation device relative to the localized target locations. Similarly, neuromodulation therapies such as deep brain stimulation can be delivered by implanting an electrode adjacent the localized target locations, or otherwise directing electrical stimulation to the localized target locations.

In some embodiments, the neuromodulation delivered can be adjusted based on the particular localized target location. For example, if a localized target location corresponds to a grayordinate associated with an integration zone where multiple functional networks interact, a different neuromodulation may be delivered to that grayordinate than to a grayordinate associated with only a single functional network. In this way, the systems and methods described in the present disclosure can provide adaptive neuromodulation therapy that reduces or otherwise avoids modulating or overmodulating functional networks that may not provide a therapeutic effect to the subject.

Additionally or alternatively, a report can be generated by the computer system based on the localized target locations, as indicated at step 224. The report can indicate a neuromodulation treatment plan for delivering the neuromodulation to the one or more target locations. The treatment plan can include one or more neuromodulation settings for controlling the operation of the neuromodulation device, such as the settings that may be delivered in step 222. Additionally or alternatively, the report can include images (e.g., medical images of the subject, the energy distribution maps) and can provide image guidance for delivering neuromodulation to the localized target locations. In still other example, the report can include data reporting on the delivery of neuromodulation to the subject, including neural activity measured in response to the delivered neuromodulation, subject feedback on preference for one or more selections of neuromodulation settings, and so on.

As one non-limiting example, subject-specific functional brain mapping and computational models of electric field propagation were used to quantify the cumulative electric field within network across functional networks. Over 60,000 different brain areas (2 mm resolution) distributed across the cortex were simulated to target and the coil position that produced the highest electric field magnitude on the target was identified in 10 subjects. For each coil position, the electric field obtaining a brain wide electric field distribution was simulated. For each target region the relative energy distributed across networks was calculated.

The energy distribution pattern was found to be subject specific, dependent on subject brain anatomy and functional organization, resulting in a subject-specific E-field pattern across networks for each individual. The salience network shows low variability in all participants, meaning a low percentage of the applied energy lies within the network. Frontoparietal network remains has low variability between participants, preserving between the 20% and 60% of the applied energy. The energy per network was quantified in 10 individuals, showing that individualized interventions can result in different results led by the specific functional brain organization and anatomy. Energy distribution maps could be very informative when evaluating intervention outcomes or side effects in individualized intervention planning.

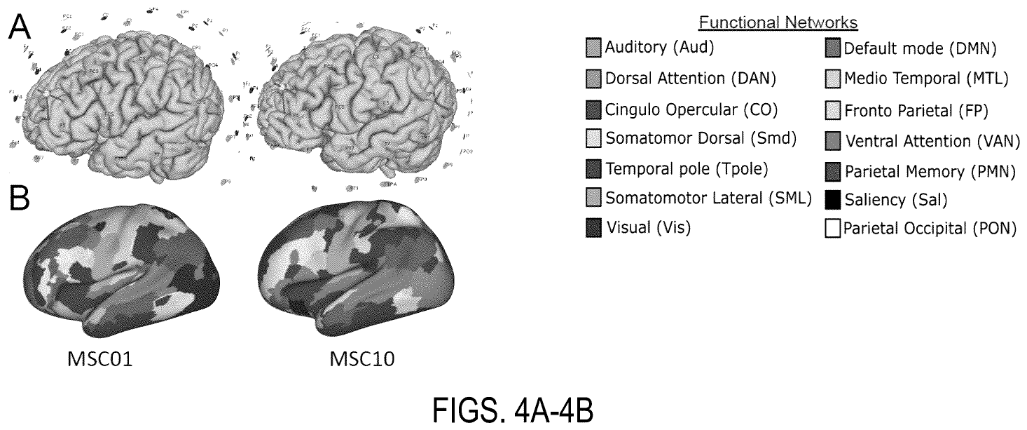

FIGS. 4A and 4B show two individual brains from a Midnight Scan Club (MSC). FIG. 4A shows a cortex with 10/20 individualized reference positions, in yellow F3, common target for DLPFC. FIG. 4B shows individualized network organization using precision functional mapping, showing the variability between individuals in functional structure across subjects.

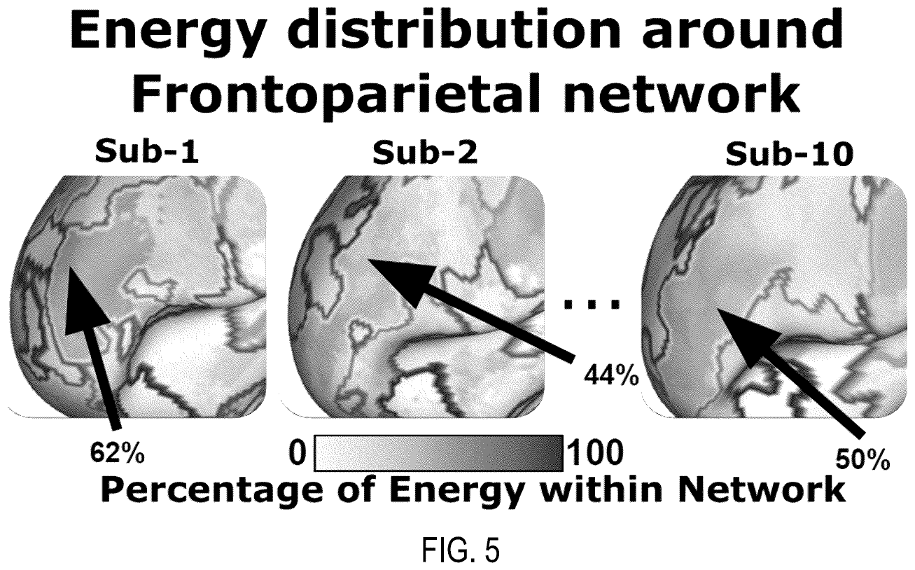

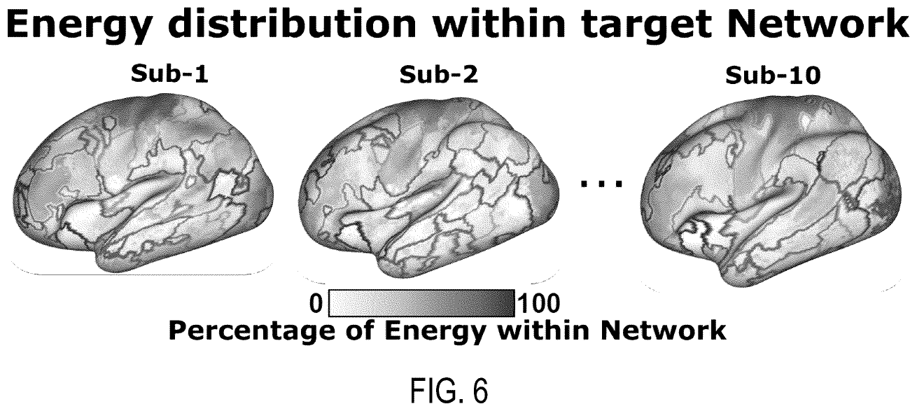

FIG. 5 shows a detail view of the energy distribution in the left frontoparietal area (yellow outline) in 3 participants of the midnight scan club dataset. Energy distribution is different among individuals with some regions within network preserving high values of energy and moderate values in some others participants. Outline colors correspond to individualized functional networks. Functional and anatomical spatial differences in energy distribution were observed. For instance, visual cortex retained high energy in all the subjects, while frontoparietal network showed more variability. FIG. 6 shows whole cortex energy distribution maps for the three individuals from the MSC dataset. Energy distribution within network was observed to vary across participants and across networks.

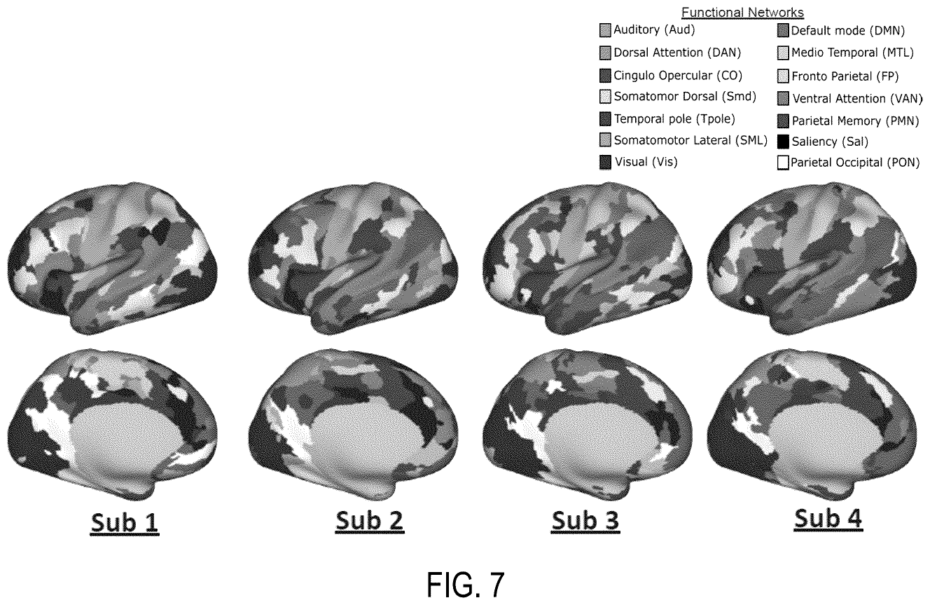

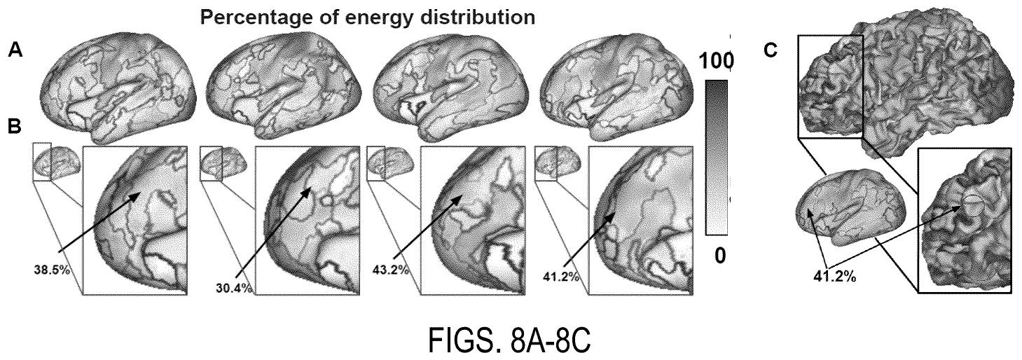

TMS treatment effectiveness in adults with TRD improves when individualized functional connectivity data is used to guide the intervention. In an example study, precision functional mapping was used to identify potential functional targets for TMS in adolescents. FIG. 7 illustrates four individual brains from an Adolescent Brain Cognitive Development (ABCD) study sample. Individualized network organization using precision functional mapping showed the variability between individuals in functional structure across subjects. It was observed that the optimal target region within a network of interest, the coil position and orientation, and the energy distributed in the target and adjacent networks can be identified using the methods described in the present disclosure. FIGS. 8A-8C show example energy distribution maps generated using the techniques described in the present disclosure. FIG. 8A illustrates the percentage of energy distribution in four participants of the ABCD sample. Energy distribution was observed to be different among individuals in some regions within network. FIG. 8B shows a detailed view in the left frontal area (yellow outline). FIG. 8C shows a detailed view on the thresholded map in one sample subject.

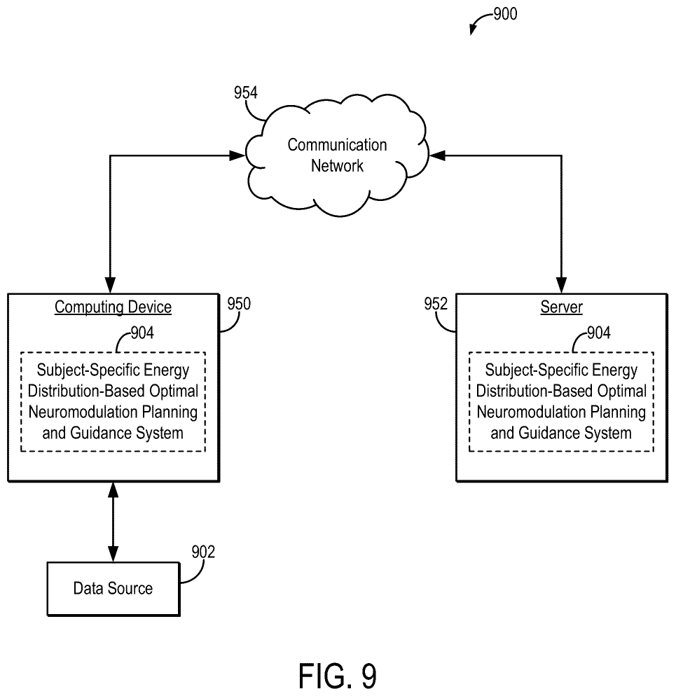

FIG. 9 shows an example of a system 900 for generating subject-specific energy distribution maps and planning and/or guiding neuromodulation therapy based on those data in accordance with some embodiments of the systems and methods described in the present disclosure. As shown in FIG. 9, a computing device 950 can receive one or more types of data (e.g., magnetic resonance image data, time course signal data, subject-specific energy distribution maps) from data source 902, which may be a magnetic resonance image source. In some embodiments, computing device 950 can execute at least a portion of a subject-specific energy distribution-based optimal neuromodulation planning and guidance system 904 to generate subject-specific energy distribution maps from data received from the data source 902 and to guide the delivery of neuromodulation therapies based on those subject-specific energy distribution maps.

Additionally or alternatively, in some embodiments, the computing device 950 can communicate information about data received from the data source 902 to a server 952 over a communication network 954, which can execute at least a portion of the subject-specific energy distribution-based optimal neuromodulation planning and guidance system. In such embodiments, the server 952 can return information to the computing device 950 (and/or any other suitable computing device) indicative of an output of the subject-specific energy distribution-based optimal neuromodulation planning and guidance system.

In some embodiments, computing device 950 and/or server 952 can be any suitable computing device or combination of devices, such as a desktop computer, a laptop computer, a smartphone, a tablet computer, a wearable computer, a server computer, a virtual machine being executed by a physical computing device, and so on. The computing device 950 and/or server 952 can also reconstruct images from the data.

In some embodiments, data source 902 can be any suitable source of image data (e.g., measurement data, images reconstructed from measurement data), such as an MRI system, another computing device (e.g., a server storing image data), and so on. In some embodiments, data source 902 can be local to computing device 950. For example, data source 902 can be incorporated with computing device 950 (e.g., computing device 950 can be configured as part of a device for capturing, scanning, and/or storing images). As another example, data source 902 can be connected to computing device 950 by a cable, a direct wireless link, and so on. Additionally or alternatively, in some embodiments, data source 902 can be located locally and/or remotely from computing device 950, and can communicate data to computing device 950 (and/or server 952) via a communication network (e.g., communication network 954).

In some embodiments, communication network 954 can be any suitable communication network or combination of communication networks. For example, communication network 954 can include a Wi-Fi network (which can include one or more wireless routers, one or more switches, etc.), a peer-to-peer network (e.g., a Bluetooth network), a cellular network (e.g., a 3G network, a 4G network, etc., complying with any suitable standard, such as CDMA, GSM, LTE, LTE Advanced, WiMAX, etc.), a wired network, and so on. In some embodiments, communication network 954 can be a local area network, a wide area network, a public network (e.g., the Internet), a private or semi-private network (e.g., a corporate or university intranet), any other suitable type of network, or any suitable combination of networks. Communications links shown in FIG. 9 can each be any suitable communications link or combination of communications links, such as wired links, fiber optic links, Wi-Fi links, Bluetooth links, cellular links, and so on.

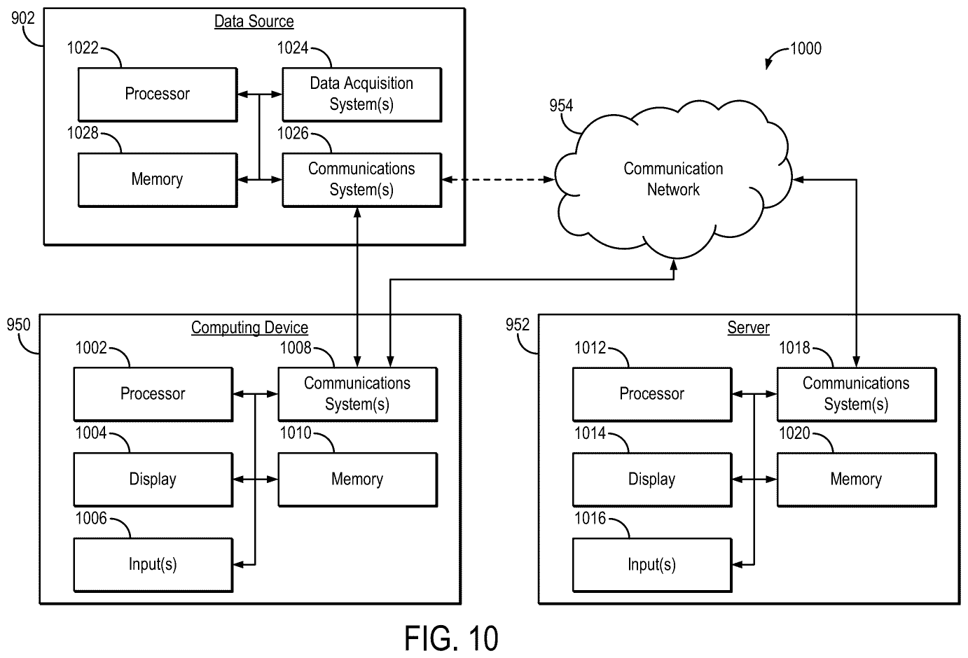

Referring now to FIG. 10, an example of hardware 1000 that can be used to implement data source 902, computing device 950, and server 952 in accordance with some embodiments of the systems and methods described in the present disclosure is shown. As shown in FIG. 10, in some embodiments, computing device 950 can include a processor 1002, a display 1004, one or more inputs 1006, one or more communication systems 1008, and/or memory 1010. In some embodiments, processor 1002 can be any suitable hardware processor or combination of processors, such as a central processing unit (“CPU”), a graphics processing unit (“GPU”), and so on. In some embodiments, display 1004 can include any suitable display devices, such as a computer monitor, a touchscreen, a television, and so on. In some embodiments, inputs 1006 can include any suitable input devices and/or sensors that can be used to receive user input, such as a keyboard, a mouse, a touchscreen, a microphone, and so on.

In some embodiments, communications systems 1008 can include any suitable hardware, firmware, and/or software for communicating information over communication network 954 and/or any other suitable communication networks. For example, communications systems 1008 can include one or more transceivers, one or more communication chips and/or chip sets, and so on. In a more particular example, communications systems 1008 can include hardware, firmware and/or software that can be used to establish a Wi-Fi connection, a Bluetooth connection, a cellular connection, an Ethernet connection, and so on.

In some embodiments, memory 1010 can include any suitable storage device or devices that can be used to store instructions, values, data, or the like, that can be used, for example, by processor 1002 to present content using display 1004, to communicate with server 952 via communications system(s) 1008, and so on. Memory 1010 can include any suitable volatile memory, non-volatile memory, storage, or any suitable combination thereof. For example, memory 1010 can include RAM, ROM, EEPROM, one or more flash drives, one or more hard disks, one or more solid state drives, one or more optical drives, and so on. In some embodiments, memory 1010 can have encoded thereon, or otherwise stored therein, a computer program for controlling operation of computing device 950. In such embodiments, processor 1002 can execute at least a portion of the computer program to present content (e.g., images, user interfaces, graphics, tables), receive content from server 952, transmit information to server 952, and so on.

In some embodiments, server 952 can include a processor 1012, a display 1014, one or more inputs 1016, one or more communications systems 1018, and/or memory 1020. In some embodiments, processor 1012 can be any suitable hardware processor or combination of processors, such as a CPU, a GPU, and so on. In some embodiments, display 1014 can include any suitable display devices, such as a computer monitor, a touchscreen, a television, and so on. In some embodiments, inputs 1016 can include any suitable input devices and/or sensors that can be used to receive user input, such as a keyboard, a mouse, a touchscreen, a microphone, and so on.

In some embodiments, communications systems 1018 can include any suitable hardware, firmware, and/or software for communicating information over communication network 954 and/or any other suitable communication networks. For example, communications systems 1018 can include one or more transceivers, one or more communication chips and/or chip sets, and so on. In a more particular example, communications systems 1018 can include hardware, firmware and/or software that can be used to establish a Wi-Fi connection, a Bluetooth connection, a cellular connection, an Ethernet connection, and so on.

In some embodiments, memory 1020 can include any suitable storage device or devices that can be used to store instructions, values, data, or the like, that can be used, for example, by processor 1012 to present content using display 1014, to communicate with one or more computing devices 950, and so on. Memory 1020 can include any suitable volatile memory, non-volatile memory, storage, or any suitable combination thereof. For example, memory 1020 can include RAM, ROM, EEPROM, one or more flash drives, one or more hard disks, one or more solid state drives, one or more optical drives, and so on. In some embodiments, memory 1020 can have encoded thereon a server program for controlling operation of server 952. In such embodiments, processor 1012 can execute at least a portion of the server program to transmit information and/or content (e.g., data, images, a user interface) to one or more computing devices 950, receive information and/or content from one or more computing devices 950, receive instructions from one or more devices (e.g., a personal computer, a laptop computer, a tablet computer, a smartphone), and so on.

In some embodiments, data source 902 can include a processor 1022, one or more inputs 1024, one or more communications systems 1026, and/or memory 1028. In some embodiments, processor 1022 can be any suitable hardware processor or combination of processors, such as a CPU, a GPU, and so on. In some embodiments, the one or more inputs 1024 are generally configured to acquire data, images, or both, and can include an MRI system. Additionally or alternatively, in some embodiments, one or more inputs 1024 can include any suitable hardware, firmware, and/or software for coupling to and/or controlling operations of an MRI system. In some embodiments, one or more portions of the one or more inputs 1024 can be removable and/or replaceable.

Note that, although not shown, data source 902 can include any suitable inputs and/or outputs. For example, data source 902 can include input devices and/or sensors that can be used to receive user input, such as a keyboard, a mouse, a touchscreen, a microphone, a trackpad, a trackball, and so on. As another example, data source 902 can include any suitable display devices, such as a computer monitor, a touchscreen, a television, etc., one or more speakers, and so on.

In some embodiments, communications systems 1026 can include any suitable hardware, firmware, and/or software for communicating information to computing device 950 (and, in some embodiments, over communication network 954 and/or any other suitable communication networks). For example, communications systems 1026 can include one or more transceivers, one or more communication chips and/or chip sets, and so on. In a more particular example, communications systems 1026 can include hardware, firmware and/or software that can be used to establish a wired connection using any suitable port and/or communication standard (e.g., VGA, DVI video, USB, RS-232, etc.), Wi-Fi connection, a Bluetooth connection, a cellular connection, an Ethernet connection, and so on.

In some embodiments, memory 1028 can include any suitable storage device or devices that can be used to store instructions, values, data, or the like, that can be used, for example, by processor 1022 to control the one or more inputs 1024, and/or receive data from the one or more inputs 1024; to images from data; present content (e.g., images, a user interface) using a display; communicate with one or more computing devices 950; and so on. Memory 1028 can include any suitable volatile memory, non-volatile memory, storage, or any suitable combination thereof. For example, memory 1028 can include RAM, ROM, EEPROM, one or more flash drives, one or more hard disks, one or more solid state drives, one or more optical drives, and so on. In some embodiments, memory 1028 can have encoded thereon, or otherwise stored therein, a program for controlling operation of data source 902. In such embodiments, processor 1022 can execute at least a portion of the program to generate images, transmit information and/or content (e.g., data, images) to one or more computing devices 950, receive information and/or content from one or more computing devices 950, receive instructions from one or more devices (e.g., a personal computer, a laptop computer, a tablet computer, a smartphone, etc.), and so on.

In some embodiments, any suitable computer readable media can be used for storing instructions for performing the functions and/or processes described herein. For example, in some embodiments, computer readable media can be transitory or non-transitory. For example, non-transitory computer readable media can include media such as magnetic media (e.g., hard disks, floppy disks), optical media (e.g., compact discs, digital video discs, Blu-ray discs), semiconductor media (e.g., random access memory (“RAM”), flash memory, electrically programmable read only memory (“EPROM”), electrically erasable programmable read only memory (“EEPROM”)), any suitable media that is not fleeting or devoid of any semblance of permanence during transmission, and/or any suitable tangible media. As another example, transitory computer readable media can include signals on networks, in wires, conductors, optical fibers, circuits, or any suitable media that is fleeting and devoid of any semblance of permanence during transmission, and/or any suitable intangible media.

As used herein in the context of computer implementation, unless otherwise specified or limited, the terms “component,” “system,” “module,” “framework,” and the like are intended to encompass part or all of computer-related systems that include hardware, software, a combination of hardware and software, or software in execution. For example, a component may be, but is not limited to being, a processor device, a process being executed (or executable) by a processor device, an object, an executable, a thread of execution, a computer program, or a computer. By way of illustration, both an application running on a computer and the computer can be a component. One or more components (or system, module, and so on) may reside within a process or thread of execution, may be localized on one computer, may be distributed between two or more computers or other processor devices, or may be included within another component (or system, module, and so on).

In some implementations, devices or systems disclosed herein can be utilized or installed using methods embodying aspects of the disclosure. Correspondingly, description herein of particular features, capabilities, or intended purposes of a device or system is generally intended to inherently include disclosure of a method of using such features for the intended purposes, a method of implementing such capabilities, and a method of installing disclosed (or otherwise known) components to support these purposes or capabilities. Similarly, unless otherwise indicated or limited, discussion herein of any method of manufacturing or using a particular device or system, including installing the device or system, is intended to inherently include disclosure, as embodiments of the disclosure, of the utilized features and implemented capabilities of such device or system.

The present disclosure has described one or more preferred embodiments, and it should be appreciated that many equivalents, alternatives, variations, and modifications, aside from those expressly stated, are possible and within the scope of the invention.

1. A method for planning, guiding, or monitoring a delivery of neuromodulation to a subject's brain, the method comprising:
(a) accessing subject-specific energy distribution mapping data with a computer system;
(b) determining one or more target locations in the subject-specific energy distribution mapping data using the computer system, wherein the one or more target locations indicate locations to which neuromodulation should be delivered; and
(c) generating target localization data by localizing the one or more target locations relative to the brain of the subject, thereby planning, guiding, or monitoring delivery of the neuromodulation to the one or more target locations.

2. The method of claim 1, wherein accessing the subject-specific energy distribution mapping data comprises accessing image data acquired from the subject and generating the subject-specific energy distribution mapping data from the image data.

3. The method of claim 2, wherein the image data comprise structural image data comprising anatomical magnetic resonance images and functional image data.

4. The method of claim 3, wherein the functional image data comprise at least one of subject-specific functional network maps or subject-specific integration zone maps.

5. The method of claim 3, wherein generating the subject-specific energy distribution mapping data comprises:
determining optimal neuromodulation positions from the image data;
generating simulated electric field data using the optimal neuromodulation positions;
calculating an energy distribution across functional networks using the simulated electric field data; and
generating the subject-specific energy distribution mapping data based on the calculated energy distribution.

6. The method of claim 5, wherein the optimal neuromodulation positions are determined from the image data by generating a cortical reconstruction from the structural image data and determining an optimal neuromodulation position over the cortical surface reconstruction for each brainordinate in each functional network of the functional image data.

7. The method of claim 6, wherein the cortical reconstruction is generated based on a finite element model.

8. The method of claim 5, wherein subject-specific energy distribution is generated by thresholding the electric field, counting a number of brainordinates within each network after thresholding, and calculating a relative energy distribution at each brainordinate as a ratio of a number of brainordinates surviving the threshold within each network and a total number of brainordinates surviving the thresholding.

9. The method of claim 5, wherein the optimal neuromodulation positions are determined from the image data using an auxiliary dipole method.

10. The method of claim 5, wherein calculating the energy distribution across the functional networks using the simulated electric field data includes projecting the simulated electric field data onto a cortical surface reconstruction generated from the image data.

11. The method of claim 1, further comprising determining neuromodulation settings based on the one or more target locations and controlling a neuromodulation device to deliver neuromodulation to the one or more target locations according to the neuromodulation settings.

12. The method of claim 1, further comprising outputting the target localization data by generating a report that indicates a neuromodulation treatment plan for delivering the neuromodulation to the one or more target locations.

13. The method of claim 1, further comprising outputting the target localization data by generating a report that indicates an image-based guidance for delivering neuromodulation to the one or more target locations.
